(19) United States
(12) Patent Application Publication
(10) Pub. No.: US 2010/0158829 A1
(43) Pub. Date: Jun. 24, 2010

(54) Method and Composition for Color Modulation

(75) Inventors: John Steven Bajor, Cheshire, CT (US); Carol Annette Bosko, Newtown, CT (US); Jorge Louie CardeNas, Milford, CT (US); Diana Jean Drennan, Naugatuck, CT (US); Diana Marrero, Bristol, CT (US); Sheila Alves Rocha, Bogota, NJ (US); Leonard Shore McGarvey, Pomona, NY (US); Margaret McGarvey, Pomona, NY (US)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc., d/b/a UNILEVER, Englewood Cliffs, NJ (US)

(21) Appl. No.: 12/343,817

(22) Filed: Dec. 24, 2008

(51) Int. Cl. A61K 8/49; A61K 38/05; A61K 31/22; A61K 31/167; A61K 31/421; A61K 31/352; A61K 31/44; A61Q 17/04

(52) U.S. Cl. 424/59; 514/19; 514/549; 514/616; 514/375; 514/456; 514/455; 514/356

(57) ABSTRACT

The invention is directed to a method and composition suitable for skin color modulation. Particularly, the action of D-dopachrome tautomerase on melanogenesis and/or the transfer of melanin from melanosomes in keratinocytes is reduced, thereby resulting in a change in skin color.

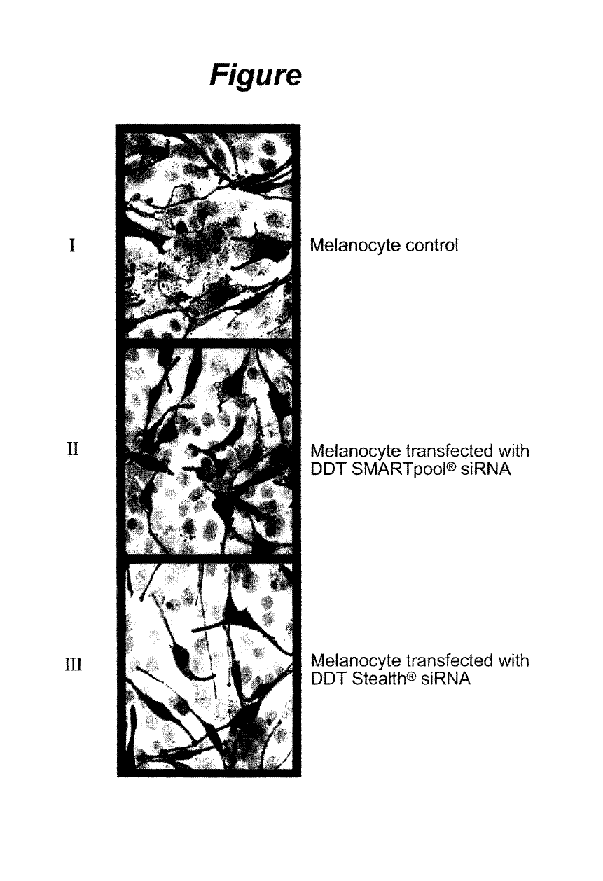

### FIELD OF THE INVENTION

The present invention is directed to a method and composition suitable to use for skin color modulation wherein it has been unexpectedly discovered that D-dopachrome tautomerase is associated with skin color. Particularly, the invention is directed to targeting D-dopachrome tautomerase and/or receptors thereof in order to, surprisingly, achieve skin color modulation. More particularly, the invention is directed to a method for modulating skin color wherein the method inhibits the affect of D-dopachrome tautomerase on melanogenesis and/or the transfer of melanin from melanosomes to keratinocytes.

### BACKGROUND OF THE INVENTION

Many consumers are concerned with the characteristics of their skin. For example, consumers with age spots or freckles often wish for such pigmented spots to be less pronounced. Other consumers may wish to reduce skin darkening caused by exposure to sunlight or to lighten their natural skin color. To meet the needs of consumers, many attempts have been made to develop products that reduce pigment production in melanocytes (i.e. reduce melanogenesis). The products developed thus far, however, often tend to have low efficacy or undesirable side effects, such as, for example, toxicity or skin irritation.

There is an increasing interest to develop a means that effectively modulates skin color, and especially, via a biological pathway. This invention, therefore, is directed to a method and cosmetic composition that inhibits the effect of D-dopachrome tautomerase on melanogenesis and/or the transfer of melanin from melanosomes to keratinocytes. The method and composition may employ a component that can, for example, inhibit the activity of D-dopachrome tautomerase and/or inhibit the binding of the same to its cognate receptor.

### Additional Information

Efforts have been disclosed for making skin care cosmetic compositions. In U.S. Pat. No. 6,875,425, skin lightening agents with 4-substituted resorcinol derivative compounds are described.

Other efforts have been disclosed for making compositions to treat skin. In U.S. Patent Nos. 7,250,158, 7,247,294 and 7,270,805, methods for treating skin with lightening agents are described.

Still other efforts have been disclosed for treating skin. In U.S. Pat. No. 5,998,423, compositions with polycyclic nitrogen heterocycles are described. U.S. Pat. No. 6,573,050 describes diagnosis and evaluation of anti-cancer therapy. The reference describes the melanocytic protein L-dopachrome tautomerase. It is known that D-dopachrome tautomerase and L-dopachrome tautomerase, known in the melanin synthesis pathway, have no homology.

Even other efforts have been concerned with dopachrome tautomerase. In U.S. Pat. No. 7,312,221, inhibitors of migration inhibitory factor are described.

None of the additional information above describes, for example, a biological pathway for modulating skin color by inhibiting the effect of D-dopachrome tautomerase on melanogenesis and/or the transfer of melanin from melanosomes to keratinocytes. Moreover, none of the additional information above describes a method for skin color modulation by targeting D-dopachrome tautomerase and/or receptors thereof.

### SUMMARY OF THE INVENTION

In a first aspect, the present invention is directed to a method for modulating skin color by targeting D-dopachrome tautomerase and/or receptors thereof.

In a second aspect, the present invention is directed to a method for modulating skin color, the method comprising the step of inhibiting the effect of D-dopachrome tautomerase on melanogenesis and/or the transfer of melanin from melanosomes to keratinocytes.

In a third aspect, the present invention is directed to a composition suitable for topical application whereby the composition upon topical application inhibits the effect of D-dopachrome tautomerase on melanogenesis and/or the transfer of melanin from melanosomes to keratinocytes.

All other aspects of the present invention will more readily become apparent upon considering the detail description and examples which follow.

Composition, as used herein, is meant to include a composition for topical application to skin of mammals, especially humans. Such a composition may be generally classified as leave-on or rinse off, and is meant to include conditioners or tonics, lipsticks, color cosmetics, and general topical compositions that in some fashion and at the very least can have an affect on skin.

Lightening, as used herein, is meant to mean the lightening of skin directly as well as the lightening of spots (hyperpigmentation) on the skin, like age spots and freckles. Such lightening can be physical and/or biological in nature, but is preferably at least biological. Modulating skin color, as used herein, means changing the color of skin but preferably skin lightening. The composition of the present invention can be in the form of a liquid, lotion, cream, foam, scrub, gel, soap bar or toner, or applied via a face mask, pad or patch. Skin as used herein is meant to include skin on the face, neck, chest, back, arms, hands, legs, buttocks and scalp. All ranges identified herein are meant to implicitly include all ranges subsumed therein if, for example, reference to the same is not explicitly made. Comprising, as used herein, includes consisting essentially of and consisting of. DDT, as used herein, is meant to mean D-dopachrome tautomerase. Component, as used herein, means an ingredient suitable for use with humans and able to inhibit the effect of D-dopachrome tautomerase on melanogenesis and/or the effect of D-dopachrome tautomerase on the transfer of melanin from melanosomes to keratinocytes (i.e., melanosome transfer). Targeting, as used herein, includes interfering with an enzyme-receptor pathway.

### BRIEF DESCRIPTION OF THE DRAWING

The subject matter which is regarded as the invention is particularly pointed out and distinctly claimed in the concluding portion of the specification. The invention, however, may be best understood by the following description taken in conjunction with the accompanying drawing figure in which:

The Figure demonstrates that inhibiting DDT will result in a reduction of melanosome transfer.

### DETAILED DESCRIPTION OF THE PREFERRED EMBODIMENTS

The only limitation with respect to the method of the present invention is that the step or steps taken to inhibit the effect of DDT on melanogenesis (and/or the transfer of melanin from melanosomes to keratinocytes) is/are suitable for use with humans. While such a method generally comprises the step of inhibiting the effect of DDT on skin darkening, the method preferably comprises the step of inhibiting the activity of DDT, inhibiting the binding of DDT to its cognate receptor, or both in order to inhibit the effect of DDT on melanogenesis and/or the effect of DDT on the transfer of melanin from melanosomes to keratinocytes. The method may be achieved, for example, by injecting, ingesting and/or topically applying a component suitable to impede the activity of DDT. In a preferred embodiment, however, the method is achieved by applying component via a topical composition, and especially, a topical cosmetic composition.

Component suitable for use in the present invention is limited only to the extent that the same may be used by humans and inhibit the effect of DDT on melanogenesis and/or the effect of DDT on melanin transfer from melanosomes to keratinocytes (e.g., suitable to interfere with an enzyme-receptor pathway). Illustrative but non-limiting examples of the types of components suitable for use in the present invention (including mixtures thereof) are represented by the formulae:

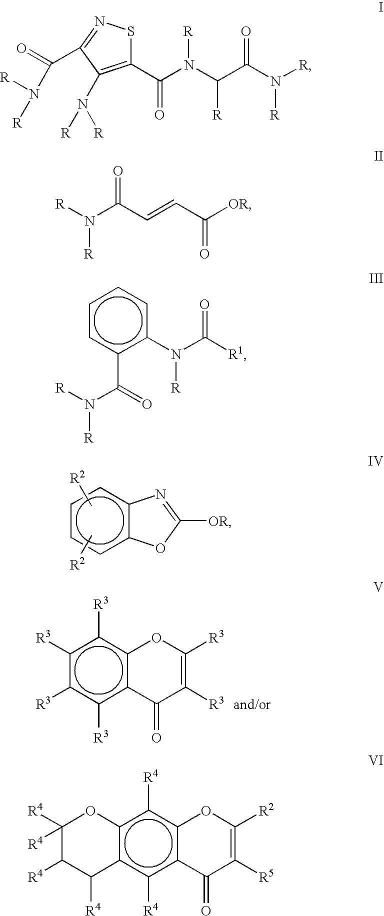

where:

each R is independently H, C₁₋₈ linear, branched or cyclic alkyl, substituted or unsubstituted aryl or heteroaryl;

R¹ is independently or a C₁₋₆ linear, branched or cyclic alkyl or substituted or unsubstituted heteroaryl;

each R² is independently H, C₁₋₆ linear, branched or cyclic alkyl or a halogen;

each R³ is independently H, C₁₋₆ linear, branched or cyclic alkyl, C₁₋₆ alkenyl, heteroalkyl;

OR⁶, N(R⁶)₂, NR⁶COR⁶, OCOR⁶ or aryl,

with the proviso that not more than two R³ groups are aryl;

each R⁴ is H, or OH;

each R⁵ is independently H, OH, or

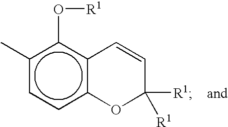

each R⁶ is independently a H or a C₁₋₆ linear, branched or cyclic alkyl.

Often preferred components suitable for use in this invention include N5-((cyclopentylcarbamoyl)(thiophen-2-yl)methyl)-4-amino-N5-phenylisothiazole-3,5-dicarboxamide, N5-((tert-butylcarbamoyl)(4-fluorophenyl)methyl)-4-amino-N5-benzylisothiazole-3,5-dicarboxamide; N5-((tert-butylcarbamoyl)(4-fluorophenyl)methyl)-4-amino-N5-phenylisothiazole-3,5-dicarboxamide; N5-((tert-butylcarbamoyl)(4-fluorophenyl)methyl)-4-amino-N5-(3-fluorophenyl)isothiazole-3,5-dicarboxamide; N5-(1-tert-butylcarbamoyl)-N5-(2-chlorobenzyl)-4-aminoisothiazole-3,5-dicarboxamide; N5-(1-cyclopentylcarbamoyl)-3-methylbutyl)-4-amino-N5-phenylisothiazole-3,5-dicarboxamide; N5-(1-cyclopentylcarbamoyl)-3-methylbutyl)-4-amino-N5-(3-fluorophenyl)isothiazole-3,5-dicarboxamide, as represented by formula I. Other preferred components suitable for use in this invention include (E)-3-(4-ethylphenylcarbamoyl)acrylic acid; (E)-3-(6-tert-butyl-3-cyano-4,5,6,7-tetrahydrobenzo[b]thiophen-2-ylcarbamoyl)acrylic acid, (E)-3(3-(ethoxycarbonyl)-6-tert-pentyl-4,5,6,7-tetrahydrobenzo[b]thiophen-2-ylcarbamoyl)acrylic acid, as represented by formula II.

N-(2-(methylcarbamoyl) phenyl)-5,6,7,8-tetrahydro-2,4-dihydroxyquinoline-3-carboxamide as represented by formula III.

7-diiodobenzo[d]oxazol-2-ol, as presented by formula IV.

Still other preferred components include, and (2-(2,4-dihydroxyphenyl)-3,5,7-4H-1-benzopyran-4-one) (Morin), dihydroxy flavone, 3-hydroxyflavone, as represented by formula V.

Even other preferred components suitable for use in this invention include 3,4-dihydro-3-hydroxy-7-(5-methoxy-2,2-dimethyl-2H-1-benzopyran-6-yl)-2,2-dimethyl-2H,6H-benzo[1,2-b:5,4-b′]dipyran-6-one (Mundulone), as represented by formula VI.

Still other components suitable for use in this invention include sodium isostearoyl lactylate, 5,6,7-trihydroxy-3-(3,4,5-trihydroxyphenyl)-4H-1-benzopyran-4-one (Irigenol), 5-hydroxy-1,4-naphthalenedione (Juglone), 1-[2R, 3R-3,5-dihydroxy-7-(3,4,5-trihydroxybenzoyl)oxychroman-2-yl]-3,5-dihydroxy-6-oxo-8-[(3R)-3,5,7-trihydroxychroman-2-yl]benzol[7]annulen-4-yl]3,4,5-trihydroxybenzoate (theaflavin digallate) or a mixture thereof.

Typically, the composition of the present invention comprises from about 0.001 to about 15%, and preferably, from about 0.02 to about 10%, and most preferably, from about 0.05 to about 5% by weight component, based on total weight of the composition and including all ranges subsumed therein.

It should be known that commercially acceptable and conventional vehicles may be used, acting as diluents, dispersants and/or carriers for the components described herein and for any other optional but often preferred additives. Therefore, the cosmetically acceptable vehicle suitable for use in this invention may be aqueous-based, anhydrous or an emulsion whereby a water-in-oil or oil-in-water emulsion is generally preferred. If the use of water is desired, water typically makes up the balance of the composition, and preferably, makes up from about 5 to about 99%, and most preferably, from about 40 to about 80% by weight of the composition, including all ranges subsumed therein.

In addition to water, organic solvents may be optionally included to act as carriers or to assist carriers within the compositions of the present invention. Illustrative and non-limiting examples of the types of organic solvents suitable for use in the present invention include alkanols like ethyl and isopropyl alcohol, mixtures thereof or the like.

Other optional additives suitable for use include ester oils like isopropyl myristate, cetyl myristate, 2-octyldodecyl myristate, avocado oil, almond oil, olive oil, neopentylglycol dicaprate, mixtures thereof or the like. Typically, such ester oils assist in emulsifying the composition of this invention, and an effective amount is often used to yield a stable, and most preferably, water-in-oil emulsion.

Emollients may also be used, if desired, as carriers within the composition of the present invention. Alcohols like 1-hexadecanol (i.e., cetyl alcohol) are often desired as are the emollients generally classified as silicone oils and synthetic esters. Silicone oils suitable for use include cyclic or linear polydimethylsiloxanes containing from 3 to 9, preferably from 4 to 5, silicon atoms. Nonvolatile silicone oils useful as an emollient material in the composition described herein include polyalkyl siloxanes, polyalkylaryl siloxanes and polyether siloxane copolymers. The essentially non-volatile polyalkyl siloxanes useful herein include, for example, polydimethylsiloxanes.

The ester emollients that may optionally be used are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
   (3) Polyhydric alcohol esters. Ethylene glycol mono and di-fatty acid esters, diethylene glycol mono-and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, stearyl stearate and arachidyl behenate.
(5) Sterol esters, of which cholesterol fatty acid esters are examples.

Emollients, when used, typically make up from about 0.1 to about 50% by weight of the composition, including all ranges subsumed therein.

Fatty acids having from 10 to 30 carbon atoms may also be included as cosmetically acceptable carriers within the composition of the present invention. Illustrative examples of such fatty acids include pelargonic, lauric, myristic, palmitic, stearic, isostearic, oleic, linoleic, arachidic, behenic or erucic acid, and mixtures thereof. Compounds that are believed to enhance skin penetration, like dimethyl sulfoxide, may also be used as an optional carrier.

Humectants of the polyhydric alcohol type may also be employed in the composition of this invention. The humectant often aids in increasing the effectiveness of the emollient and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycerol or sodium hyaluronate. The amount of humectant may range anywhere from 0.2 to 25%, and preferably, from about 0.5 to about 15% by weight of the composition, based on total weight of the composition and including all ranges subsumed therein.

Thickeners may also be utilized as part of the cosmetically acceptable carrier in the composition of the present invention. Typical thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0 to 5%, usually from 0.001 to 1%, optimally from 0.01 to 0.5% by weight.

Collectively, the water, solvents, silicones, esters, fatty acids, humectants and/or thickeners will constitute the cosmetically acceptable carrier in amounts from 1 to 99.9%, preferably from 80 to 99% by weight.

Surfactants may also be present in the composition of the present invention. Total concentration of the surfactant will range from about 0 to about 40%, and preferably, from about 0 to about 20%, optimally from about 0 to about 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; mono- and di- fatty acid esters of ethylene glycol; fatty acid monoglyceride; sorbitan, mono- and di- C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

Perfumes may be used in the composition of this invention. Illustrative non-limiting examples of the types of perfumes that may be used include those described in Bauer, K., et al., Common Fragrance and Flavor Materials, VCH Publishers (1990).

Illustrative yet non-limiting examples of the types of fragrances that may be used in this invention include myrcene, dihydromyrenol, citral, tagetone, cis-geranic acid or citronellic acid, mixtures thereof or the like.

Preferably, the amount of fragrance employed in the composition of this invention is in the range from about 0.0% to about 10%, more preferably, about 0.00001% to about 5 wt %, most preferably, about 0.0001% to about 2%.

Various types of optional active ingredients may be used in the compositions of the present invention. Actives are defined as skin benefit agents other than emollients and other than ingredients that merely improve the physical characteristics of the composition. Although not limited to this category, general examples include talcs and silicas, as well as alpha-hydroxy acids, beta-hydroxy acids, peroxides, zinc salts, sunscreens, natural products and/or extracts.

Beta-hydroxy acids include salicylic acid, for example. Zinc pyrithione is an example of the zinc salts useful in the skin lightening composition of the present invention.

Sunscreens include those materials commonly employed to block ultraviolet light. Illustrative compounds are the derivatives of PABA, cinnamate and salicylate. For example, avobenzophenone (Parsol 1789®), octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation. Additives that reflect or scatter the suns rays may also be employed. These additives include oxides like zinc oxide and titanium dioxide.

Many topical compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Still other optional active ingredients that may be used with the composition of this invention include dioic acids (e.g., malonic acid, sebacic acid), antioxidants like vitamin E, other vitamins, like vitamin C and its derivatives, tyrosinase inhibitors, like hydroquinone, resorcinols and their derivatives (including those esterified with, for example, ferulic acid, vanillic acid or the like) and retinoids, including retinoic acid, retinal, retinol and retinyl esters, conjugated linoleic acid, petroselinic acid and mixtures thereof, as well as any other conventional ingredients well known for wrinkle-reducing, skin whitening (especially, niacinamide and/or cis-en dicycloether), anti-acne effects and reducing the impact of sebum.

The composition of the present invention is intended for use primarily as a cosmetic product for topical application to human skin, especially and at least as a product for lightening the skin. Thus, the inventors have discovered that D-dopachrome tautomerase is, unexpectedly, associated with pigment production, and inhibiting the activity of D-dopachrome tautomerase can result in desirable skin lightening benefits and especially, when the compositions of this invention are applied topically to areas of the skin where lightening or whitening is desired. Other benefits may include skin moisturizing, decreasing the effect of sebum on the skin and skin wrinkle reducing. In an especially preferred embodiment, the composition of the present invention has a pH from about 4.5 to about 7.5, including all ranges subsumed therein.

When making the composition of the present invention, the desired ingredients are mixed, in no particular order, and usually at temperatures from about ambient to about 80° C. and under atmospheric pressure.

The packaging for the composition of this invention can be, for example, a patch, bottle, tube, roll-ball applicator, propellant driven aerosol device, pump, twist or squeeze container, lidded jar or stick.

The examples which follow are provided to illustrate and facilitate an understanding of the invention. The examples are not intended to limit the scope of the claims.

### EXAMPLE 1

Melanosome transfer was assessed by using a coculture assay. Human epidermal melanocytes were grown to 60-80% confluency. The cells were transfected with siRNA (small interfering RNA) according to the Sequitur, Inc., art recognized and available protocol then overlaid with the HaCaT keratinocytes after 72 hours. The cocultures were incubated in keratinocyte growth medium (like the medium made available by PromoCell GmbH) without phorbol ester, and were fixed 48 hours post-overlay for detection of melanin pigment by the Fontana-Masson staining procedure. Slides were visualized using a light microscope (Carl Zeiss MicroImaging GmbH) and images were acquired using art recognized AxioVision software. Ten random high power fields were analyzed per coculture sample.

Coculture experiments to test for melanosome transfer in melanocytes transfected with DDT siRNA demonstrated that there was a decrease in melanosome transfer after DDT knockdown (inhibition), surprisingly suggesting that DDT plays a role in melanosome transfer and pigmentation in keratinocytes. The results in the Figure at I show normal melanosome transfer in a positive control for DDT. Melanocytes transfected with DDT SMARTpool® and Stealth® siRNA (shown in the Figure at II and at III, respectively) reduced transfer, as reflected in less staining of cells at 48 hours post-coculture (120 hour post-transfection). The Stealth® and SMARTpool® siRNA were made commercially available by Invitrogen and Millipore, respectively. The results confirm the surprising discovery that targeting DDT and/or receptors thereof results in skin color modulation.

### EXAMPLE 2

### Preparation of D-Dopachrome Substrate and Assay

A 125-mM sodium periodate working solution was prepared in H₂O in an amber 5-mL vial (prepared fresh on the day of the assay). 4.3 mg of dihydroxy-D-phenylalanine (D-dopa) were weighed into a 20 mL scintillation vial equipped with a pierceable septa screw cap. With the use of a liquid transfer cannula, 12 mL of degassed buffer was first transferred into a 15 mL graduated conical tube and subsequently poured into the scintillation vial containing the D-dopa. The D-dopa was dissolved for ten (10) minutes while stirring under nitrogen. The resulting working solution was 1.8 mM. Once sodium periodate was added to the dissolved D-dopa, the resulting D-dopachrome was utilized immediately to prevent substrate degradation.

Concentrations were based on a final reaction volume of 200 μL. A total of 75 μL of buffer (100 mM potassium phosphate, pH 6.8) was added to each well of a clear flat bottom 96-well microtiter plate. To the blank control wells (no DDT), 25 μL of 10% glycerol/assay buffer was added. DMSO (5 μL) was added to blank wells and to the enzyme control wells (DDT alone with no inhibitors). Positive control wells contained 75 μM arachidonic acid (final concentration) which gives an inhibition of approximately 50%. To the remaining wells, 5 μL of test compounds diluted in DMSO was added for a final concentration of 100 μM. Lastly, 25 μL of DDT in assay buffer was added to all wells except for the substrate control wells. The concentration of DDT utilized was such that it produced a ΔOD₄₇₅ of approximately 1.0 at the 40-second read time point between the blank and total.

With the use of a 1 mL syringe, 0.33 ml of the 125 mM sodium periodate working solution was added to the dissolved D-dopa and stirred for 30 seconds. The resulting D-dopachrome was transferred to a reagent reservoir and 100 μL immediately added to the first row of interest in the assay plate (resulting final substrate concentration in the assay is 0.9 mM). The plate was shaken for 2 seconds on a Spectromax plate reader and the decrease in absorbance measured over 40 seconds. 100 μL of D-dopachrome was added to the next row of interest in the assay plate and this procedure was repeated until four (4) rows were assayed and read. Often approximately 6-7 minutes, the remaining substrate was discarded.

Percent inhibition was calculated utilizing the OD values at the 40-second time point according to the following formula:

Percent inhibition=((total-unknown)/(total-blank))*100 For Data Normalization; Normalized percent inhibition for unknown=(A)*(B/C)

### Where:

A=percent inhibition of the unknown;
B=average percent inhibition for the 4 positive control values (within 4 distinct assay rows) generated with a single batch of substrate also used to assay the unknown of interest;
C=percent inhibition for the positive control for the assay row of interest which contains the unknown compound of interest.

### EXAMPLE 3

A stock solution of 10 mM L-dopa (3,4-Dihydroxyphenylalanine, Sigma Cat #D9628) was prepared in sodium phosphate buffer (100 mM, pH 7.0) along with a stock solution of 0.1 mg/mL (605 units/ml) mushroom tyrosinase (Sigma cat #T7755) in phosphate buffer and stored at room 4° C. until use.

Test compounds (dissolved in DMSO) were first diluted in phosphate buffer to working concentrations of 1 mM. For each test, 150 μL of phosphate buffer, 10 μL of the L-dopa stock and 20 μL of each compound were added to each well of a 96 well clear bottom microtiter plate and mixed three times. 20 μL of mushroom tyrosinase stock solution was added, mixed and the absorbency read at 475 nm at 0, 2, 4, and 6.5 minutes. The points were plotted as absorbency vs. time and the slope of the line calculated. Values are expressed as the percent of the respective untreated control reaction. The final DMSO concentration in each sample was less than or equal to 1.0%

TABLE

| | D-Dopachrome | Mushroom |
|---|---|---|
| | Tautomerase at | Tyrosinase at |
| | 100 μM | 100 μM |
| Component | (% of control) | (% of control) |
| N5- | 66.8% | 101.8% |
| ((cyclopentylcarbamoyl)(thiophen- | | |
| 2-yl)methyl)-4-amino-N5- | | |
| phenylisothiazole-3,5- | | |
| dicarboxamide | | |
| N5-((tert-butylcarbamoyl)(4- | 69.9% | 98.6% |
| fluorophenyl)methyl)-4-amino-N5- | | |
| benzylisothiazole-3,5- | | |
| dicarboxamide | | |
| N5-((tert-butylcarbamoyl)(4- | 52.3% | 101.9% |
| fluorophenyl)methyl)-4-amino-N5- | | |
| phenylisothiazole-3,5- | | |
| dicarboxamide | | |
| N5-((tert-butylcarbamoyl)(4- | 48.0% | 106.1% |
| fluorophenyl)methyl)-4-amino-N5- | | |
| (3-fluorophenyl)isothiazole-3,5- | | |
| dicarboxamide | | |
| N-5-(1-(tert- | 64.3% | 102.5% |
| butylcarbamoyl)butyl)-N5-(2- | | |
| chlorobenzyl)-4-aminoisothiazole- | | |
| 3,5-dicarboxamide | | |
| N5-(1-(cyclopentylcarbamoyl)-3- | 69.2% | 89.4% |
| methylbutyl)-4-amino-N5- | | |
| phenylisothiazole-3,5- | | |
| dicarboxamide | | |
| N5-(1-(cyclopentylcarbamoyl)-3- | 64.9% | 105.2% |
| methylbutyl)-4-amino-N5-(3- | | |
| fluorophenyl)isothiazole-3,5- | | |
| dicarboxamide | | |
| N5-(1-(cyclopentylcarbamoyl)-3- | 65.7% | 100.6% |
| methylbutyl)-4-amino-N5-(4- | | |
| fluorophenyl)isothiazole-3,5- | | |
| dicarboxamide | | |
| N5-((tert-butylcarbamoyl)(1H- | 40.9% | 96.0% |
| indol-3-yl)methyl)-4-amino-N5- | | |
| phenylisothiazole-3,5- | | |
| dicarboxamide | | |
| 5,6-diiodobenzo[d]oxazol-2-ol | 56.1% | 99.3% |
| N-(2-(methylcarbamoyl)phenyl)- | 65.3% | 100.0% |
| 5,6,7,8-tetrahydro-2,4- | | |
| dihydroxyquinoline-3-carboxamide | | |
| (E)-3-(4- | 40.5% | 89.4% |
| ethylphenylcarbamoyl)acrylic acid | | |
| (E)-3-(6-tert-butyl-3-cyano- | 8.4% | 88.2% |
| 4,5,6,7- | | |
| tetrahydrobenzo[b]thiophen-2- | | |
| ylcarbamoyl)acrylic acid | | |
| (E)-3-(3-(ethoxycarbonyl)-6-tert- | 14.9% | 90.8% |
| pentyl-4,5,6,7- | | |
| tetrahydrobenzo[b]thiophen-2- | | |
| ylcarbamoyl)acrylic acid | | |
| 4-Ethyl Resorcinol | 100% | 42.3% |
| | (100 μM) | (Tested at 1 μM) |

The results demonstrate that the components did not inhibit tyrosinase at a final concentration of 100 μM as was the case for 4-ethyl resorcinol, a potent inhibitor of mushroom tyrosinase and inactive in the DDT assay. Surprisingly, it was discovered that the components ellicit their inhibiting effects on melanogenesis by inhibiting DDT.

### EXAMPLE 4

Melanoderm™ tissue equivalent model MEL-300 (MatTek: Ashland, Mass.) containing melanocytes obtained from dark skin individuals were cultured as per supplier instructions. Components were added to the maintenance medium phase (no topical treatments) at a final concentration of 10 μM for 14 days at which time the experiment was terminated and the tissues assessed for melanin content. Medium and treatments were changed three times per week. DMSO was utilized as the vehicle control and all treatments were performed in duplicate.

For quantification of total melanin, each Melanoderm was placed into an individual Eppendorf tube (2 mL) into which 250 μL of Solvable™ tissue solubilizer (Packard Bioscience Co., Cat#6NE9100) was added. Each tube was vortexed and incubated at 60° C. for 18 hours. After about 12 hours, each sample was centrifuged (5-minutes at 13,000 RPMs) to remove any particulates. One hundred μL of supernatant were transferred to a 96 well microtiter plate and the absorbance read at 490 nm. Total melanin content was calculated from a standard curve using synthetic melanin (Sigma Cat#M8631).

All the following components were tested at a final concentration of 10 μM:

Juglone: 32.8% Reduction in Melanin; IC₅₀ 14.9 μM

Theaflavin Digallate: 26.6% Reduction in Melanin; IC₅₀ 19.6 μM

Irigenol: 7.0% Reduction in Melanin; IC₅₀ 37.4 μM

Sodium Isostearoyl Lactylate: 6.2% Reduction in Melanin; IC₅₀ 14.6 μM

Morin 7.2% Reduction in Melanin; IC₅₀ 27.4 μM

All IC₅₀ values are taken from DDT assays similar to those described in Example 2.

The results show that components consistent with this invention inhibit melanogenesis.

What is claimed is:

1. A method for modulating skin color comprising the step of inhibiting the effect of DDT on melanogenesis and/or the transfer of melanin from melanosomes to keratinocytes.

2. The method according to claim 1 wherein modulating skin color comprises the lightening of skin, age spots, freckles and/or spots resulting from skin hyperpigmentation.

3. The method according to claim 1 wherein inhibiting the effect of DDT is achieved by inhibiting the activity of DDT.

4. The method according to claim 1 wherein inhibiting the effect of DDT is achieved by inhibiting the binding of DDT to its receptor.

5. The method according to claim 1 wherein the method further comprises the step of delivering a component suitable to inhibit the effect of DDT.

6. The method according to claim 5 wherein the component is delivered with an injection, by ingesting or by topically applying the component.

7. The method according to claim 6 wherein the component is topically applied in a cosmetic composition.

8. A method for modulating skin color comprising the step of targeting DDT and/or receptors thereof to interfere with a DDT-receptor pathway.

9. The method according to claim 8 wherein the DDT-receptor pathway is interfered with by applying a topical composition.

10. A composition comprising:
a) a component suitable to inhibit the effect of DDT on melanogenesis and/or the transfer of melanin from melanosomes to keratinocytes; and
b) a cosmetically acceptable carrier.

11. The composition according to claim 10 wherein the component inhibits the activity of DDT, inhibits the binding of DDT to its receptor, or both.

12. The composition according to claim 10 wherein the composition is delivered to a consumer by injection, ingestion or topically.

13. The composition according to claim 12 wherein the composition is delivered to a consumer in a topical cosmetic composition.

14. The composition according to claim 10 wherein the component is at least one of: 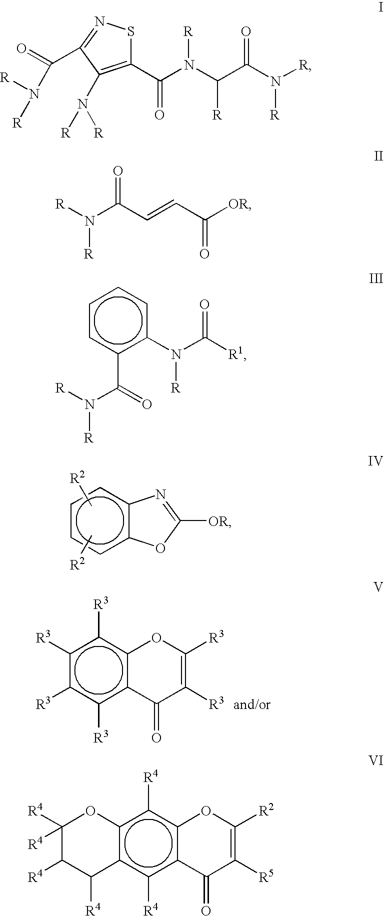
where:
each R is independently H, C₁₋₈ linear, branched or cyclic alkyl, substituted or unsubstituted aryl or heteroaryl;
R¹ is independently or a C₁₋₆ linear, branched or cyclic alkyl or substituted or unsubstituted heteroaryl;
each R² is independently H, C₁₋₆ linear, branched or cyclic alkyl or a halogen;
each R³ is independently H, C₁₋₆ linear, branched or cyclic alkyl, C₁₋₆ alkenyl, heteroalkyl,
OR⁶, N(R⁶)₂, NR⁶COR⁶, OCOR⁶ or aryl,
with the proviso that not more than two R³ groups are aryl,
each R⁴ is H, or OH;
each R⁵ is independently H, OH, or 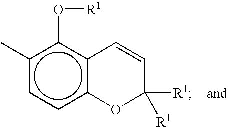
each R⁶ is independently a H or a C₁₋₆ linear, branched or cyclic alkyl.

15. The composition according to claim 10 wherein the composition comprises from about 0.001 to about 15% by weight component.

16. The composition according to claim 10 wherein the composition further comprises niacinamide.

17. The composition according to claim 10 wherein the composition further comprises an alpha-hydroxy acid, beta-hydroxy acid, sunscreen, fragrance, petroselinic acid, conjugated linoleic acid or a mixture thereof.

18. The composition according to claim 10 wherein the component is sodium isostearoyl lactylate, 5,6,7-trihydroxy-3-(3,4,5-trihydroxyphenyl)- 4H-1-benzopyran-4-one, 5-hydroxy-1,4-naphthalenedione, 1-[2R, 3R-3,5-dihydroxy-7-(3,4,5-trihydroxybenzoyl)oxychroman-2-yl]-3,5-dihydroxy-6-oxo-8-[(3R)-3,5,7-trihydroxychroman-2-yl]benzol[7]annulen-4-yl]3,4,5-trihydorxybenzoate or a mixture thereof.

19. The composition according to claim 10 wherein the composition further comprises a tyrosinase inhibitor.
